(19) United States
(12) Patent Application Publication
(10) Pub. No.: US 2022/0387267 A1
(43) Pub. Date: Dec. 8, 2022

(54) FINE BUBBLE-CONTAINING COSMETIC FOR IMPROVING DERMAL PENETRATION, MANUFACTURING SYSTEM OF FINE BUBBLE-CONTAINING COSMETIC FOR IMPROVING DERMAL PENETRATION, AND MANUFACTURING METHOD OF FINE BUBBLE-CONTAINING COSMETIC FOR IMPROVING DERMAL PENETRATION

(71) Applicant: Sung-Joo Lee, Seoul (KR)

(72) Inventor: Sung-Joo Lee, Seoul (KR)

(21) Appl. No.: 17/777,175

(22) PCT Filed: Nov. 3, 2020
(86) PCT No.: PCT/KR2020/015200

§ 371 Date: May 16, 2022

(30) Foreign Application Priority Data
Nov. 22, 2019 (KR) 10-2019-0151414

(51) Int. Cl. A61K 8/04; A61K 8/19; A61Q 19/00

(52) U.S. Cl. CPC A61K 8/046; A61K 8/19; A61Q 19/00; A61K 2800/10

(57) ABSTRACT

Provided are a cosmetic solution of which ingredients are absorbed through a skin of a human body, a fine bubble-containing cosmetic for improving skin penetration which includes the fine bubbles contained in the cosmetic solution to be absorbed rapidly, and a system and a method for manufacturing the cosmetic.

The fine bubble-containing cosmetic for improving dermal penetration of the present invention contains fine bubbles to allow maximization of penetration ability of the cosmetic into a skin.

### TECHNICAL FIELD

The present invention relates to a cosmetic which may contain fine bubbles to improve dermal penetration ability, a system for manufacturing the cosmetic, and a method for use in cosmetic manufacture.

### BACKGROUND ART

As shown in FIG. 1, skin is largely composed of epidermis S1, corium S2, and subcutaneous tissue S3, the epidermis includes stratum corneum 1 and a new skin layer 2, and a sweat gland 4, a skin pore 5, and a sebaceous gland 6 are formed on the corium 3, and when a cosmetic is applied to the skin, the cosmetic penetrates a lipid layer 1-2 formed between corneocytes 1-1 forming the stratum corneum 1, as shown in FIG. 2, or is absorbed into a new skin layer 2, a stratum basal 6 positioned between the new skin layer 2 and the corium 3, the corium S2, and the subcutaneous tissue S3 through the sweat gland 4, the sebaceous gland 6, and the like.

Here, the place where absorption is most actively performed is a lipid layer 1-2, and since the lipid layer 1-2 sensitively responds to skin irritation, when minute skin irritation is applied to the lipid layer 1-2, cosmetic penetration ability through the lipid layer 1-2 may be increased.

Conventionally, in order to increase the cosmetic penetration ability, a method of irritating the skin using a hand in the course of applying the cosmetic or irritating the skin using special equipment was used, but since this method requires separate time and equipment for skin irritation, the method had time constraints and space constraints.

Accordingly, there is a need for a new cosmetic for solving the time constraints and space constraints.

### RELATED ART DOCUMENTS

### Patent Documents

KR 2019-0110887 A (Oct. 1, 2019)

### Disclosure of Invention

### Technical Problem

An object of the present invention is to provide a cosmetic for increasing dermal penetration ability of the cosmetic by irritating skin.

Another object of the present invention is to provide a manufacturing method and a manufacturing system for increasing the cosmetic effect.

### Solution to Problem

In one general aspect, a fine bubble-containing cosmetic for improving dermal penetration includes: a cosmetic solution of which ingredients are absorbed through a skin of a human body; and fine bubbles contained in the cosmetic solution.

The fine bubbles may be formed of a plurality of gas particles having a particle diameter of 0.06 Mm to 10 Mm.

The gas particles may include any one or more of oxygen, air, hydrogen, and nitrogen.

In another general aspect, a manufacturing system of the fine bubble-containing cosmetic for improving dermal penetration includes: an ultrapure water storage unit storing a base solution used in cosmetic manufacture; one or more composition storage units storing cosmetic raw materials mixed with the base solution stored in the ultrapure water storage unit; one or more agitators mixing the base solution and the cosmetic raw materials to form a cosmetic composition; a cosmetic storage unit storing the manufactured cosmetic; a viscosity measurement unit measuring a viscosity of the cosmetic composition formed in the one or more agitators; a fine bubble formation positioning unit comparing a viscosity measured in the viscosity measurement unit and a stored optional viscosity to determine the fine bubble formation position; and a fine bubble formation unit forming the fine bubbles in the position determined in the fine bubble formation positioning unit.

The fine bubble formation positioning unit may form the fine bubbles in the ultrapure water storage unit or the agitator positioned before the agitator with a measured viscosity among the one or more agitators when a viscosity numerical value measured in the viscosity measurement unit is more than 150% based on a viscosity of ultrapure water at the same temperature, and may form the fine bubbles in the cosmetic storage unit or the agitator positioned after the agitator with a measured viscosity among the one or more agitators when a viscosity numerical value measured in the viscosity measurement unit is 150% or less based on a viscosity of ultrapure water at the same temperature.

The manufacturing system may further include a condition control unit controlling any one or more of a temperature, a humidity, and a pressure of the base solution or the cosmetic composition introduced to the agitator.

The manufacturing system may further include an impurity removal unit removing impurities from the cosmetic composition transferred to the cosmetic storage unit.

In still another general aspect, a manufacturing method of the fine bubble-containing cosmetic for improving dermal penetration includes: preparing a base solution to prepare a base solution used in manufacture of a cosmetic; forming a composition to mix the base solution prepared in the preparation of the base solution with cosmetic raw materials to form a cosmetic composition; identifying a viscosity to identify the viscosity of the cosmetic composition formed in the formation of the composition; and determining a fine bubble formation point to determine the fine bubble formation point based on the viscosity identified in the identification of the viscosity.

In the step of determining a fine bubble formation point, when the viscosity of the cosmetic composition identified in the step of identifying a viscosity is more than 150% based on ultrapure water at the same temperature, the fine bubbles may be formed before mixing the finally mixed cosmetic raw materials.

When the viscosity of the cosmetic composition identified in the step of identifying a viscosity is 150% or less based on the viscosity of ultrapure water at the same temperature, it may be determined that the fine bubbles are formed in the cosmetic composition mixed with the final cosmetic raw materials.

### Advantageous Effects of Invention

The fine bubble-containing cosmetic for improving dermal penetration of the present invention contains fine bubbles to allow maximization of penetration ability of the cosmetic into a skin.

In detail, the tine bubbles contained in the cosmetic irritates the skin to allow the cosmetic to be absorbed more rapidly in the skin.

Further, a particle diameter of the fine bubbles is limited to a size to irritate the skin more effectively, thereby further increasing a skin care effect of the cosmetic.

In addition, since a point of mixing the fine bubbles is controlled in response to a viscosity of the cosmetic to be manufactured, fine bubble formation may be more actively performed.

### BRIEF DESCRIPTION OF DRAWINGS

The above and other objects, features and advantages of the present invention will become apparent from the following description of preferred embodiments given in conjunction with the accompanying drawings, in which:

FIG. 1 is a conceptual diagram showing skin layers of a human body.

FIG. 2 is a plan view showing stratum corneum in the skin layers of a human body.

FIG. 3 is a conceptual diagram showing a fine bubble-containing cosmetic for improving dermal penetration according to the present invention.

FIG. 4 is a graph in which an effect of a fine bubble-containing cosmetic and an effect of a cosmetic having no fine bubble are compared.

FIG. 5 is a table in which an effect of a fine bubble-containing cosmetic and an effect of a cosmetic having no fine bubbles are compared.

FIG. 6 is a graph in which a floating velocity and a Brownian motion velocity of fine bubbles are compared.

FIG. 7 is a graph showing percentages of buoyancy and Brownian motion depending on particle diameters of fine bubbles.

FIGS. 8 and 9 are tables showing changes in bubble floating velocity with respect to particle diameters of fine bubbles.

FIG. 10 is a conceptual diagram showing a manufacturing system of a fine bubble-containing cosmetic for improving dermal penetration.

FIG. 11 is a flow chart showing a manufacturing method of a fine bubble-containing cosmetic for improving dermal penetration.

### BEST MODE FOR CARRYING OUT THE INVENTION

Advantages and features of the exemplary embodiments of the present invention and methods to achieve them will be elucidated from exemplary embodiments described below in detail with reference to the accompanying drawings. However, the present invention is not limited to exemplary embodiments disclosed below, but will be implemented in various forms. The exemplary embodiments of the present invention make disclosure of the present invention thorough and are provided so that those skilled in the art can easily understand the scope of the present invention. Therefore, the present invention will be defined by the scope of the appended claims. Throughout the description, like reference numerals denote like elements.

When it is determined that the detailed description of the known functions or configurations may obscure the gist of the present invention in the description of the exemplary embodiments of the present invention, the detailed description thereof will be omitted. Further, the following terminologies are defined in consideration of the functions in the exemplary embodiments of the present invention and may be construed in different ways by the intention of users and operators or the custom. Therefore, the definitions thereof should be construed based on the contents throughout the specification.

Hereinafter, the fine bubble-containing cosmetic (C) for improving dermal penetration according to the present invention will be described with reference to the attached drawings, and bubbles shown in the data illustrated in the drawings are defined as having the same concept as the gas particles described in the present invention.

In FIG. 3, a conceptual diagram of a cosmetic case (H) in which the fine bubble-containing cosmetic (C) for improving dermal penetration according to the present invention is housed.

Referring to FIG. 3, the fine bubble-containing cosmetic for improving dermal penetration may be formed of a cosmetic solution 10 which passes through the skin of a human body to absorb ingredients and fine bubbles 20 contained in the cosmetic solution 10.

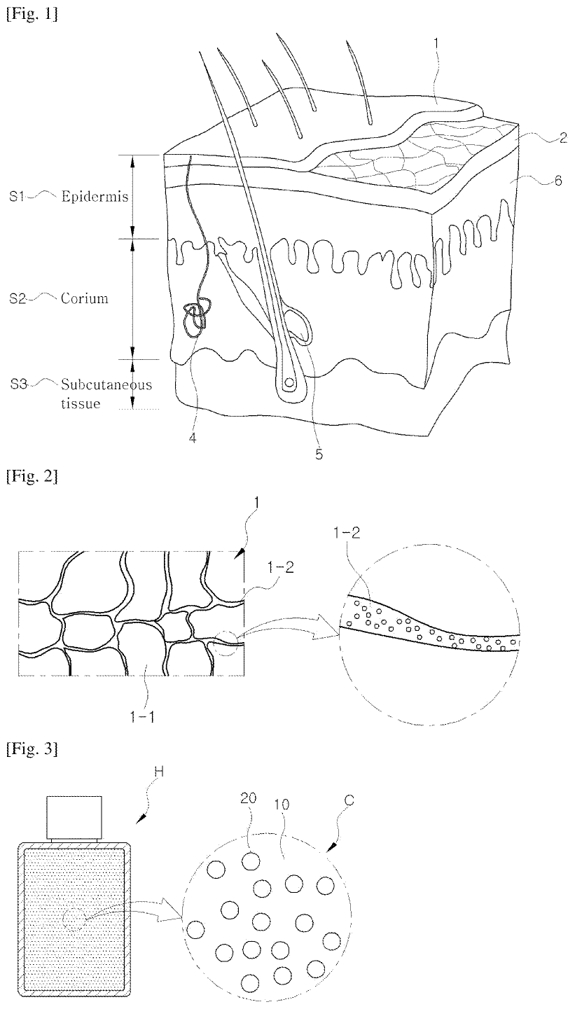

In detail, as described above with reference to FIGS. 1 and 2, the cosmetic is absorbed most through a lipid layer 1-2 formed between stratum corneum among many components forming the skin and the amount passing through the lipid layer 1-2 varies with the state of the lipid layer 1-2, and thus, in the present invention, the cosmetic solution 10 contains fine bubbles 20, so that when a user applies a cosmetic (C) to the skin, the fine bubbles 20 contained in the cosmetic solution 10 are attached to and break the stratum corneum and the lipid layer by random motion by Brownian motion to irritate the lipid layer, thereby serving to increase a penetration ratio of effective ingredients of the cosmetic.

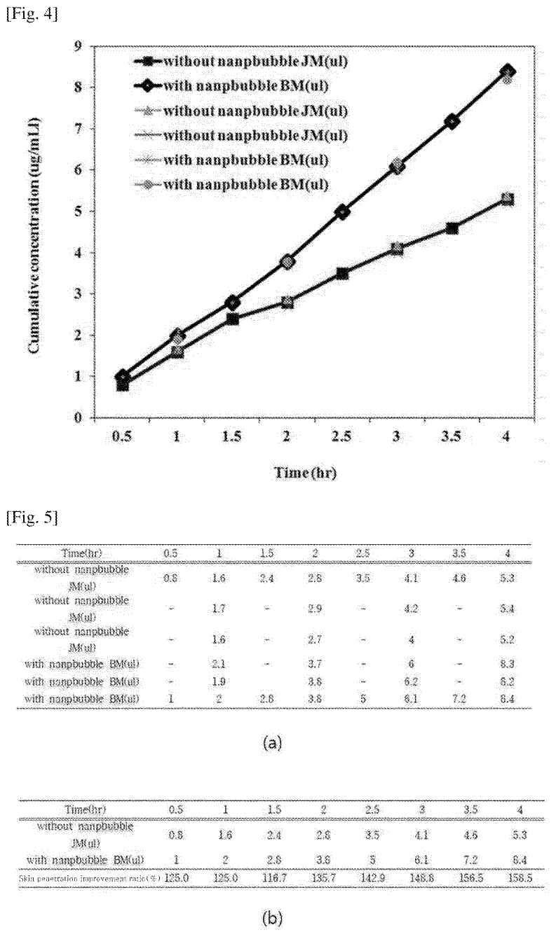

In FIG. 4, a graph of three control groups representing dermal penetration of a cosmetic solution 10 shown when fine bubbles 20 were not added to the cosmetic solution 10 and three experimental groups representing dermal penetration of the cosmetic solution 10 shown when the fine bubbles 20 were added to the cosmetic solution 10 is shown, in (a) of FIG. 5, a table representing dermal penetration over time of the three experimental groups and the three control groups is shown, and in (b) of FIG. 5, a table in which average values of the control groups and the experimental groups were calculated, respectively, and then improvement ratios of dermal penetration based thereon are compared is shown.

Referring to FIGS. 4 and 5, it was found that when the fine bubbles 20 were added to the cosmetic solution 10, the improvement ratio of dermal penetration was increased as compared with the cosmetic solution 10 to which the fine bubbles 20 were not added as time went on after applying the cosmetic. In detail, since the fine bubbles 20 are attached to or detached from the lipid layer 102 formed between corneocytes or broken to continuously apply minute irritation to the lipid layer 102, an irritation effect is slowly and consistently shown over time, while the cosmetic to which no fine bubbles 20 were added may not have the effect. Further, since the fine bubble-containing cosmetic irritates the lipid layer 1-2 with very minute irritation, people with sensitive skin may use the cosmetic without reluctance.

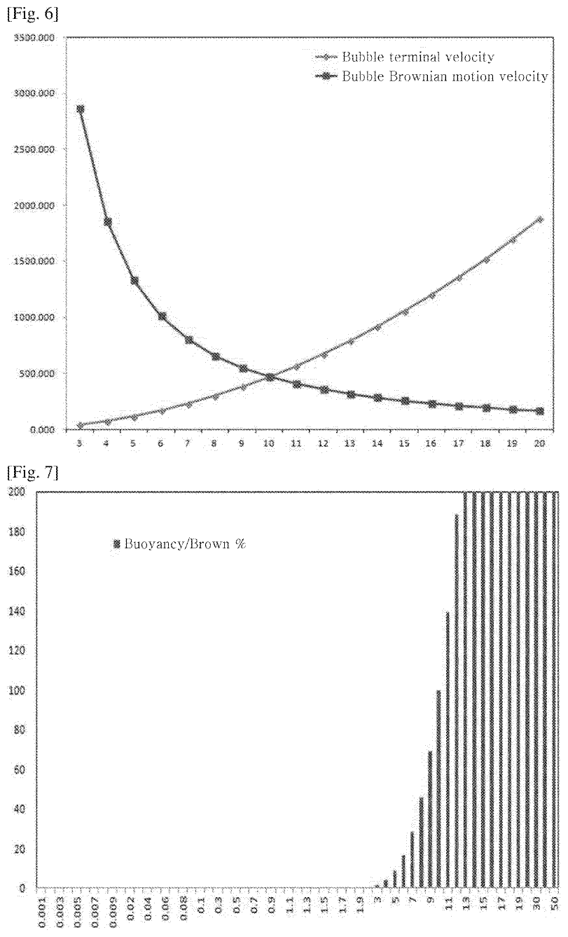

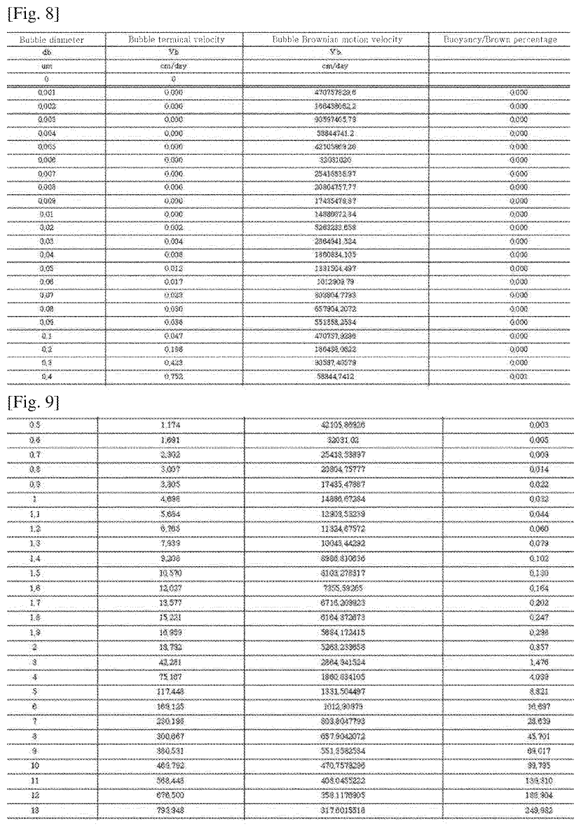

In FIG. 6, a graph in which a floating (terminal) velocity and a Brownian motion velocity of the fine bubbles 20 by buoyancy are compared is shown, in FIG. 7, a graph showing percentages of buoyancy and Brownian motion depending on particle diameters of the fine bubbles 20 is shown, and in FIGS. 8 and 9, tables showing changes in bubble floating (terminal) velocity with respect to particle diameters of the fine bubbles 20 are shown.

Referring to FIGS. 6 to 9, the fine bubbles 20 may be formed of gas particles 21 having various particle diameters, but in order to maintain a state in which the fine bubbles 20 are contained for a longer time in the cosmetic solution 10, it is recommended that the particle diameter of the gas particles 21 is 0.06 Mm or more and less than 10 Mm, and the more optimized particle diameter may be 0.1 Mm to 0.3 Mm.

In detail, a remaining time of the fine bubbles 20 contained in the cosmetic solution 10 is determined by a floating velocity of the gas particles 21 present in the state of fine particle state, and the floating velocity of the gas particles 21 is in the form of intersecting with the Brownian motion velocity of the gas particles 21. That is, the higher the Brownian motion velocity, the slower the floating velocity of the gas particles 21, so that a time during which the gas particles 21 are contained in the cosmetic solution 10 in a bubble state is increased, and the Brownian motion velocity and the floating velocity of the gas particles 21 are determined by the particle diameter of the gas particles 21, and thus, in the present invention, the particle diameter of the gas particles 21 is determined as a size at which the gas particles may remain in the cosmetic solution 10 for a longer time.

Here, the smaller the particle diameter of the gas particles 21, the higher the Brownian motion velocity and the slower the floating velocity, so that the fine bubbles 20 may be contained in the cosmetic solution 10 for a long time, but when the particle diameter of the gas particles 21 forming the fine bubbles 20 is less than 0.6 Mm, the gas particles 21 may be dissolved in the cosmetic solution 10, and thus, it is recommended that the particle diameter of the gas particles 21 is 0.6 Mm or more, and as shown in FIG. 7, when the particle diameter of the gas particles 21 is 10 Mm, the Brownian motion velocity and the floating velocity are in equilibrium with each other, and thus, it is recommended that the particle diameter is 10 Mm or less at which the Brownian motion velocity has a greater influence on the motion of the gas particles 21.

That is, by determining the particle diameter of the gas particles 21 as 0.6 μm or more and less than 10 μm, the gas particles 21 are not dissolved in the cosmetic solution 10 and the Brownian motion dominates the movement of the gas particles 21 so that floating (terminal) of the gas particles 21 occurs slowly.

Further, referring to FIGS. 8 and 9, it is recommended that the more proper particle diameter of the gas particles 21 is 0.1 Mm to 0.3 Mm. In detail, since a complicated process is needed for making the particle diameter of the gas particles 21 a certain level or less, a problem of being expensive may occur, and thus, the size of the gas particles 21 was set to a range in which the particle diameter of the gas particles 21 may be easily controlled by a mechanical method and the effect of the floating velocity of the gas particles on the Brownian motion velocity is minimized.

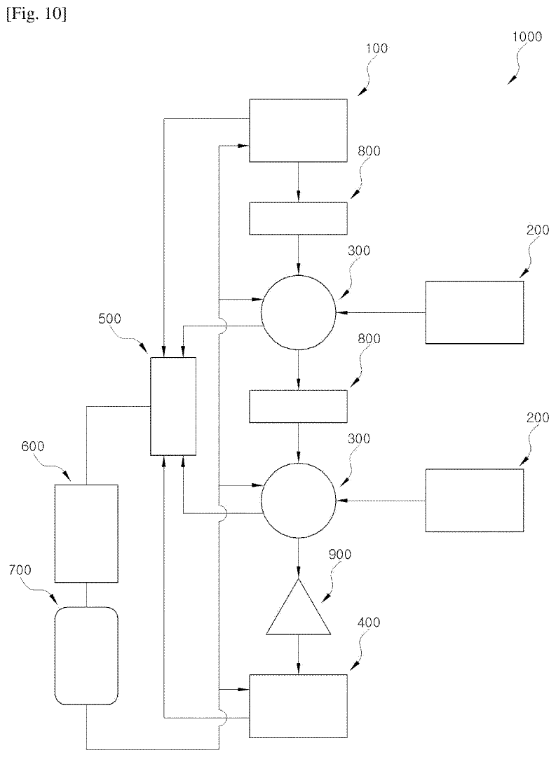

In FIG. 10, a conceptual diagram showing a manufacturing system of a fine bubble-containing cosmetic for improving dermal penetration is shown.

Referring to FIG. 10, a manufacturing system 1000 of a fine bubble-containing cosmetic for improving dermal penetration may include an ultrapure water storage unit 100 storing a base solution used in cosmetic manufacture, a composition storage unit 200 storing cosmetic raw materials mixed with the base solution stored in the ultrapure water storage unit 100, an agitator 300 mixing the base solution and the cosmetic raw materials to form a cosmetic composition, a cosmetic storage unit 400 storing the manufactured cosmetic, a viscosity measurement unit 500 measuring a viscosity of the cosmetic composition formed in the agitator 300, a fine bubble formation positioning unit 600 comparing a viscosity measured in the viscosity measurement unit 500 and a stored optional viscosity to determine the fine bubble formation position, a fine bubble formation unit 700 forming the fine bubbles in the position determined in the fine bubble formation positioning unit 600, the condition control unit 800 controlling any one or more of the temperature, humidity, and pressure of the base solution or the cosmetic composition introduced to the agitator, and an impurity removal unit 900 removing impurities in the cosmetic composition transferred to the cosmetic storage unit 400.

In detail, manufacture of the cosmetic starts with manufacture of the base solution used in the cosmetic manufacture such as pure water in the ultrapure water storage unit 100, the base solution in the ultrapure water storage unit 100 is sequentially mixed with the cosmetic raw materials stored in the composition storage unit 200 in the agitator 300 to gradually change into the cosmetic composition having similar properties to the cosmetic to be manufactured, and when all cosmetic raw materials in the cosmetic composition are mixed and have the same properties as the cosmetic to be manufactured, the cosmetic is transferred to the cosmetic storage unit 400, injected into a container, and sold.

Further, in order that the cosmetic composition have the same properties as the cosmetic to be manufactured in the process of manufacturing the cosmetic by mixing the base solution with the cosmetic raw materials, conditions such as the temperature, humidity, and pressure of the base solution, the cosmetic raw materials, and the cosmetic composition are controlled to predetermined optional numerical values in the condition control unit 800, and from the cosmetic composition made by mixing the final cosmetic raw materials in the agitator 300, impurities produced in the mixing process are removed by passing the cosmetic composition through the impurity removal unit 900 such as a membrane. That is, the cosmetic is manufactured through various processes such as mixing, chemical environmental condition control, and impurity removal.

The cosmetic manufactured through the process have various viscosities depending on the purpose, and when the viscosity is high, it may be difficult to form fine-sized gas particles 21 forming the fine bubbles in the cosmetic, and when the fine bubbles are formed in the base solution having a low viscosity, the fine bubbles may be broken, the number of the fine bubbles may be decreased, or coarsening may occur in the course of going through various processes for cosmetic manufacture. Thus, in the present invention, the viscosity of the cosmetic composition made in the agitator 300 is measured by the viscosity measurement unit 500, the manufacturing processes of the cosmetic which should be gone through in the fine bubble formation positioning unit 600 are minimized, and simultaneously, an optimized fine bubble formation position having a viscosity at which the fine bubbles may be effectively mixed, and then the fine bubbles are allowed to be formed in the determined position in the fine bubble formation unit 700.

Here, the method of forming the fine bubbles in the fine bubble formation unit 700 may be a method in which the base solution, the cosmetic composition, and the manufactured cosmetic are made collide with a specific object positioned in the atmosphere in the course of circulating the base solution, the cosmetic solution, and the manufactured cosmetic, a method of spraying the circulated composition in the atmosphere and making the composition collide with each other to capture the fine bubble particles between the sprayed particles, or a method of further injecting a fine bubble particle-containing solution to the base solution, the cosmetic composition, and the manufactured cosmetic to allow the fine bubbles to be contained therein, and there are various other methods, and thus, the method is not limited.

Further, the fine bubble formation positioning unit 600 may form the fine bubbles in the ultrapure water storage unit 100 or the agitator 300 positioned before the agitator 300 with a measured viscosity among the one or more agitators 300 when a viscosity numerical value measured in the viscosity measurement unit 500 is more than 150% based on a viscosity of ultrapure water at the same temperature, and may form the fine bubbles in the cosmetic storage unit 400 or the agitator 300 positioned after the agitator 300 with a measured viscosity among the one or more agitators 300 when a viscosity numerical value measured in the viscosity measurement unit 500 is 150% or less based on a viscosity of ultrapure water at the same temperature.

In detail, as described above, in the fine bubble formation, there is a problem that the fine bubbles are not mixed well when the viscosity of the object for forming the fine bubbles is at or above a certain level, and thus, the viscosity of the object is limited to 1.5 centipoise at which the tine bubbles may be mixed well, and based thereon, the fine bubbles are formed in the rear end of the process in which the viscosity is appropriate and the least number of the processes may be gone through.

In other words, referring to FIG. 10, when the viscosity of the cosmetic stored in the cosmetic storage unit 400 after final manufacture is less than 1.5 centipoise, the fine bubbles are directly formed in the cosmetic stored in the cosmetic storage unit 400, when the viscosity of the cosmetic stored in the cosmetic storage unit 400 is 1.5 centipoise or more, the fine bubbles are formed in the cosmetic composition positioned in the agitator 300 positioned before mixing the cosmetic composition which is finally mixed, and when the concentration of the cosmetic composition positioned in the agitator adjacent to the ultrapure water storage unit 100 is 1.5 centipoise or more, the fine bubbles are formed in the base solution stored in the ultrapure water storage unit.

Here, as the fine bubbles are formed in the initial step of the cosmetic process, the number of fine bubbles is decreased and coarsening occurs while the fine bubbles go through many processes, and thus, in response, the particle diameter of the fine bubbles should be small and the number of the fine bubbles should be increased, of course.

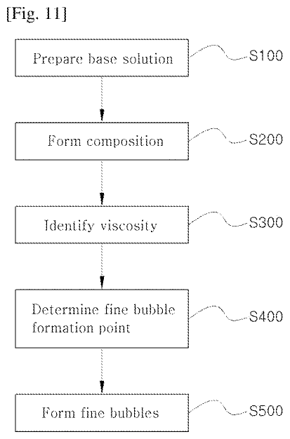

In FIG. 11, a manufacturing method of a fine bubble-containing cosmetic for improving dermal penetration is shown.

Referring to FIG. 11, a manufacturing method of a fine bubble-containing cosmetic S1000 may include a step S100 of preparing a base solution to prepare a base solution used in manufacture of a cosmetic, a step S200 of forming a composition to mix the base solution prepared in the step S100 of preparing a base solution with cosmetic raw materials to form a cosmetic composition, a step S300 of identifying a viscosity to identify the viscosity of the cosmetic composition formed in the step S200 of forming a composition, a step S400 of determining a fine bubble formation point to determine the fine bubble formation point based on the viscosity identified in the step S300 of identifying a viscosity, and a step S500 of forming fine bubbles to form the fine bubbles at the determined point.

In detail, as described above for the manufacturing system of a fine bubble-containing cosmetic for improving dermal penetration, the fine bubbles have a problem that the number of the fine bubbles is decreased and coarsening occurs as materials contained changes into a cosmetic and more processes are gone through, and when the viscosity of the subject material is at or above a certain level, it is difficult to form the fine bubbles, and thus, in the step S400 of determining a fine bubble formation point, when the viscosity of the cosmetic composition identified in the step S300 of identifying a viscosity is more than 150%, it is determined that the fine bubbles are formed before mixing the finally mixed cosmetic raw material, and when the viscosity of the cosmetic composition identified in the step S300 of identifying a viscosity is 150% or less based on the viscosity of ultrapure water at the same temperature, it is determined that the fine bubbles are formed in the cosmetic composition mixed with the final cosmetic raw material.

Also, in the present invention, various gases may be applied to the gas particles, and as an example, the gas particles are formed by hydrogen to have a skin aging prevention effect (antiaging effect), when the gas particles are formed by oxygen, oxygen is supplied to skin cells to have an effect of activating the cells, and the air and other gases may have an effective component penetration effect through irritation. That is, various gases to further increase the effect may form the gas particles depending on the cosmetic efficacy and purpose.

The present invention is not limited to the above-mentioned exemplary embodiments but may be variously applied, and may be variously modified by those skilled in the art to which the present invention pertains without departing from the gist of the present invention claimed in the claims.

### DETAILED DESCRIPTION OF MAIN ELEMENTS

10: cosmetic solution

20: fine bubble

21: gas particle

100: ultrapure water storage unit

200: composition storage unit

300: agitator

400: cosmetic storage unit

500: viscosity measurement unit

600: fine bubble formation positioning unit

700: fine bubble formation unit

800: condition control unit

900: impurity removal unit

S100: preparing base solution

S200: forming composition

S300: identifying viscosity

S400: determining fine bubble formation point

s500: forming fine bubbles

1. A fine bubble-containing cosmetic for improving dermal penetration, the cosmetic comprising:
a cosmetic solution of which ingredients are absorbed through a skin of a human body; and
fine bubbles contained in the cosmetic solution.

2. The fine bubble-containing cosmetic for improving dermal penetration of claim 1, wherein the fine bubbles are formed of a plurality of gas particles having a particle diameter of 0.06 Mm to 10 Mm.

3. The fine bubble-containing cosmetic for improving dermal penetration of claim 2, wherein the gas particles include any one or more of oxygen, air, hydrogen, and nitrogen.

4. A manufacturing system of the fine bubble-containing cosmetic for improving dermal penetration of claim 1, the manufacturing system comprising:
an ultrapure water storage unit storing a base solution used in cosmetic manufacture;
one or more composition storage units storing cosmetic raw materials mixed with the base solution stored in the ultrapure water storage unit;
one or more agitators mixing the base solution and the cosmetic raw materials to form a cosmetic composition;
a cosmetic storage unit storing the manufactured cosmetic;
a viscosity measurement unit measuring a viscosity of the cosmetic composition formed in the one or more agitators;
a fine bubble formation positioning unit comparing a viscosity measured in the viscosity measurement unit and a stored optional viscosity to determine the fine bubble formation position; and
a fine bubble formation unit forming the fine bubbles in the position determined in the tine bubble formation positioning unit.

5. The manufacturing system of a fine bubble-containing cosmetic for improving dermal penetration of claim 4, wherein the fine bubble formation positioning unit
forms the fine bubbles in the ultrapure water storage unit or the agitator positioned before the agitator with a measured viscosity among the one or more agitators when a viscosity numerical value measured in the viscosity measurement unit is more than 150% based on a viscosity of ultrapure water at the same temperature, and
forms the fine bubbles in the cosmetic storage unit or the agitator positioned after the agitator with a measured viscosity among the one or more agitators when a viscosity numerical value measured in the viscosity measurement unit is 150% or less based on the viscosity of ultrapure water at the same temperature.

6. The manufacturing system of a fine bubble-containing cosmetic for improving dermal penetration of claim 4, further comprising: a condition control unit controlling any one or more of a temperature, a humidity, and a pressure of the base solution or the cosmetic composition introduced to the agitator.

7. The manufacturing system of a fine bubble-containing cosmetic for improving dermal penetration of claim 4, further comprising: an impurity removal unit removing impurities from the cosmetic composition transferred to the cosmetic storage unit.

8. A manufacturing method of the fine bubble-containing cosmetic for improving dermal penetration of claim 1, the manufacturing method comprising:
preparing a base solution to prepare a base solution used in manufacture of a cosmetic;
forming a composition to mix the base solution prepared in the preparing of a base solution preparation with cosmetic raw materials to form a cosmetic composition;
identifying a viscosity to identify the viscosity of the cosmetic composition formed in the forming of a composition; and
determining a fine bubble formation point to determine the fine bubble formation point based on the viscosity identified in the identifying of a viscosity.

9. The manufacturing method of a fine bubble-containing cosmetic for improving dermal penetration of claim 8, wherein in the determining of a fine bubble formation point,
when the viscosity of the cosmetic composition identified in the identifying of a viscosity is more than 150% based on a viscosity of ultrapure water at the same temperature, it is determined that the fine bubbles are formed before mixing the finally mixed cosmetic raw materials, and
when the viscosity of the cosmetic composition identified in the identifying of a viscosity is 150% or less based on the viscosity of ultrapure water at the same temperature, it is determined that the fine bubbles are formed in the cosmetic composition mixed with the final cosmetic raw materials.
